(19) United States
(12) Patent Application Publication
(10) Pub. No.: US 2010/0055725 A1
(43) Pub. Date: Mar. 4, 2010

(54) SYSTEM FOR ASSAYS OF AMINOTRANSFERASE

(75) Inventors: Jianghong Rao, Sunnyvale, CA (US); Daniel Sobek, Portola Valley, CA (US)

Correspondence Address: LAW OFFICES OF MIKIO ISHIMARU, 333 W. EL CAMINO REAL, SUITE 330, SUNNYVALE, CA, 94087, US

(73) Assignee: ZYMERA, INC., Portola Valley, CA (US)

(21) Appl. No.: 12/440,677

(22) PCT Filed: Oct. 17, 2007
(86) PCT No.: PCT/US2007/081694

§ 371 Date: Mar. 10, 2009

Related U.S. Application Data
(60) Provisional application No. 60/829,874, filed on Oct. 17, 2006.
(60) Provisional application No. 60/829,876, filed on Oct. 17, 2006.

(51) Int. Cl. C12Q 1/48

(52) U.S. Cl. 435/15

(57) ABSTRACT

An assay system (300) comprising providing a platform (102) having a contact surface (104); immobilizing an amino acid group (108), having an amine side group (109), on the contact surface (104); transforming the amine side group (109), in the amino acid group (108) to a ketone group (120); and reacting an indicator (122) with the ketone group (120) for displaying a light emission (128) from the indicator (122).

### CROSS-REFERENCE TO RELATED APPLICATION(S)

This application claims the benefit of U.S. Provisional Patent Application Ser. No. 60/829,874 filed Oct. 17, 2006 and U.S. Provisional Patent Application Ser. No. 60/829,876 filed Oct. 17, 2006.

### TECHNICAL FIELD

The present invention relates generally to enzyme activity assays, and more particularly to an assay system for aminotransferases.

### BACKGROUND ART

Several heart and liver diseases have been correlated with abnormally high levels of aspartate aminotransferase (AST) present in the blood. Examples of such conditions include pulmonary embolism, viral and toxic hepatitis, acute myocardial infarction, acute pancreatitis, and acute cirrhosis. Similarly, human alanine aminotransferase (ALT) is an enzyme that may be leaked into the blood of a patient suffering from hepatic diseases such as viral hepatitis, hepatocirrhosis, and is used as a key biological marker.

Diagnosis of serum containing ALT and AST provides a good indicator of whether a particular patient is undergoing distress due to a disease. Liver or heart disease may present elevated AST or ALT levels as an indicator. The assays for determining the activity of these enzymes generally involve extracting blood from the patient and immediately employing one of a number of calorimetric or kinetic ultraviolet techniques.

Regardless of the assay format employed to determine aminotransferase activity, it has been common practice to use these assays on venous blood drawn from the patient in a clinical setting. The assays may be performed on serum or plasma separated from the whole blood drawn from the patient. This is due to the fact that hemoglobin content from red blood cells interferes with most measurements. Thus, it is preferable to remove the red blood cells from whole blood in order to avoid excessive light absorption from this protein.

In addition, aminotransferase enzyme activity in the serum is relatively unstable as a function of time, and, for this reason, it has been common practice to analyze serum or plasma relatively quickly once the serum or plasma is separated from whole blood. This practice has meant that serodiagnosis for indications of disorders in which the aminotransferase activities are elevated have been performed in the clinical setting as opposed to a setting distant from the hospital.

Aminotransferases are enzymes that catalyze the transfer of an amino group from a donor co-substrate into an acceptor co-substrate, 2-Oxoglutarate, forming L-glutamate as one of the products of the enzymatic reaction. L-Aspartate is the amino group donor co-substrate for the reaction catalyzed by the Aspartate Aminotransferase (AST) enzyme, and L-Alanine is the amino group donor for the reaction catalyzed by Alanine Aminotransferase (ALT).

Both ALT and AST require the presence of pyridoxal-5′-phosphate (P-5′-P), a protein derived from vitamin B6, as a co-enzyme. This protein attaches to the apoenzyme (i.e., an aminotransferase without this protein) and forms the active site that transfers the amine group from one co-substrate to the other. Blood contains aminotransferases with P-5-P′ and without this coenzyme.

Standard methods for the quantification of aminotransferase activity employ secondary enzymatic reactions that provide an observable change in absorbance. The enzyme-substrate system for the secondary reaction must be abundant enough such that the two-step reaction rate is limited by the first step. More specifically, the most common detection method involves employing enzymes that use nicotinamide-adenine dinucleotide (NADH) as a co-substrate for the enzymatic reduction of the oxo-acid products of the first reaction. The progression of the reaction is monitored as a decrease in absorbance at 339-340 nm created by the consumption of the NADH co-substrate. However, absorption spectroscopy is not as sensitive as fluorometry or luminescence. For example, luminescence measurements are approximately 100 times more sensitive than absorption measurements.

Thus, a need still remains for an assay system for alanine aminotransferase and aspartate aminotransferase that employs fluorescent or bioluminescent labels that are compatible with complex biological fluids. In view of the ever-increasing activity in the biosciences, it is increasingly critical that answers be found to these problems. In view of our aging population having an increase in the occurrence of heart and liver disease, it is increasingly critical that answers be found to these problems. Additionally, the need to save costs, improve efficiencies and performance, and meet competitive pressures, adds an even greater urgency to the critical necessity for finding answers to these problems.

Solutions to these problems have been long sought but prior developments have not taught or suggested any solutions and, thus, solutions to these problems have long eluded those skilled in the art.

### DISCLOSURE OF THE INVENTION

The present invention provides an assay system including providing a platform having a contact surface; immobilizing an amino acid group, having an amine side group, on the contact surface; transforming the amine side group, in the amino acid group to a ketone group; and reacting an indicator with the ketone group for displaying a light emission from the indicator.

Certain embodiments of the invention have other aspects in addition to or in place of those mentioned above. The aspects will become apparent to those skilled in the art from a reading of the following detailed description when taken with reference to the accompanying drawings.

### BRIEF DESCRIPTION OF THE DRAWINGS

FIGS. 1A, 1B, and 1C are diagrams of a system for assay of aminotransferase, in an embodiment of the present invention;

FIGS. 2A, 2B, and 2C are diagrams of a system for assay of aminotransferase, in an alternative embodiment of the present invention; and

FIG. 3 is a flow chart of a system for assays of aminotransferase for operating the system for assays of aminotransferase in an embodiment of the present invention.

### BEST MODE FOR CARRYING OUT THE INVENTION

The following embodiments are described in sufficient detail to enable those skilled in the art to make and use the invention. It is to be understood that other embodiments would be evident based on the present disclosure, and that process or mechanical changes may be made without departing from the scope of the present invention.

In the following description, numerous specific details are given to provide a thorough understanding of the invention. However, it will be apparent that the invention may be practiced without these specific details. In order to avoid obscuring the present invention, some well-known system configurations and process steps are not disclosed in detail. Likewise, the drawings showing embodiments of the system are semi-diagrammatic and not to scale and, particularly, some of the dimensions are for the clarity of presentation and are shown greatly exaggerated in the drawing FIGs. Where multiple embodiments are disclosed and described, having some features in common, for clarity and ease of illustration, description, and comprehension thereof, similar and like features one to another will ordinarily be described with like reference numerals.

For expository purposes, the term “horizontal” as used herein is defined as a plane parallel to the plane or surface of the substrate, regardless of its orientation. The term “vertical” refers to a direction perpendicular to the horizontal as just defined. Terms, such as “above”, “below”, “bottom”, “top”, “side” (as in “sidewall”), “higher”, “lower”, “upper”, “over”, and “under”, are defined with respect to the horizontal plane. The term “on” means there is direct contact among elements. The term “system” as used herein means and refers to the method and to the apparatus of the present invention in accordance with the context in which the term is used.

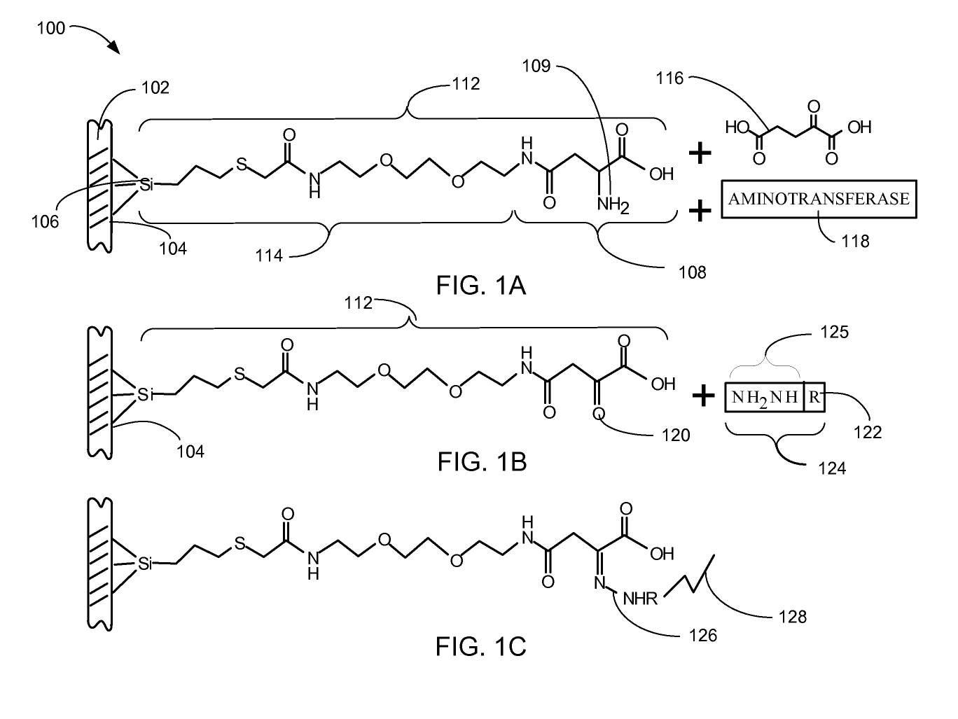

Referring now to FIGS. 1A, B, and 1C, therein is shown a diagram of a system for assay of aminotransferase 100, in an embodiment of the present invention. The diagram of the system for assay of aminotransferase 100 depicts a platform 102, such as a plastic, silicon dioxide, glass or other non-biological platform, having a contact surface 104. Attachment sites 106, such as embedded ions of the platform material, may be disbursed across the contact surface 104 or restricted to a specific region of the contact surface 104. An amino acid group 108, such as L-aspartate or L-alanine, may be immobilized by attaching it to the platform 102. The amino acid group 108 is a protein amino acid found in all forms of life. The amino acid group 108 may be a dicarboxyl amino acid found in small amounts in body fluids.

In this embodiment, a forward aspartate aminotransferase (AST) catalyzed reaction is measured by first immobilizing the amino acid group 108 on the contact surface 104 using a spacer molecule 114. A surface-linked enzyme substrate 112, having an aspartate molecule or an alinine molecule, includes a spacer molecule 114 and the amino acid group 108. The surface-linked enzyme substrate 112 is then exposed to a co-substrate (2-oxaglutarate) 116 mixed with the specimen containing an aminotransferase enzyme 118, such as aspartate aminotransferase (AST) or an alanine aminotransferase (ALT).

Referring now to FIG. 1B, therein is shown a diagram of the system for assay of aminotransferase 100, in an intermediate step of the present invention. The diagram of the system for assay of aminotransferase 100 depicts the product of the reaction catalyzed by the aminotransferase enzyme 118, which catalyzes the transfer of the amine side-group 109 in the surface-linked enzyme substrate 112 to the co-substrate 116 in the solution leaving a ketone group 120 in place of the amine side-group 109. As a second step, the surface-linked enzyme substrate 112, with a ketone group 120, is exposed to a hydrazine indicator conjugate 124 containing an indicator 122 with a hydrazine side group 125, NH2NHR, where R denotes a molecule of the indicator 122. The indicator 122 may be a chemiluminescent molecule, bioluminescent molecule, an organic dye, a luminescent nanocrystal, or a conjugate between the bioluminescent molecule and the luminescent nanocrystal.

Referring now to FIG. 1C, therein is shown a diagram of the system for assay of aminotransferase 100, in a finishing step of the present invention. The diagram of the system for assay of aminotransferase 100 depicts the hydrazine indicator conjugate 124 having reacted with the ketone group 120 on the surface-linked enzyme substrate 112, binding the indicator 122 to the surface-linked enzyme substrate 112 by a hydrazone bond 126. Following a wash step to remove any of the indicator 122 that may be unbound, a light emission 128, such as a fluorescence of luminescence emission, from the indicator 122 that remains bound to the surface-linked enzyme substrate 112 is measured and correlated to the activity of the aminotransferase enzyme 118.

This approach to the system for assay of aminotransferase enables detection of AST and ALT within whole blood, plasma, serum or other biological fluids. Embodiments where the light emission 128 occurs without an external source of illumination are well suited for handheld instrument designs. This approach may be implemented in any format including microscope slides, arrays, well plates, microfluidic channels, filters, porous materials and any combination thereof.

Alternatively the transaminase reactions may be measured by immobilizing L-glutamate on the surface and measuring the transaminase catalyzed conversion of the glutamate into surface-linked 2-oxaglutarate by labeling the ketone groups in this molecule following the approach described in the prior example. The immobilization of one of the co-substrates in the surface enables the implementation of the measurement on spots or array of spots pre-aligned to the appropriate detection optics and detectors.

In one implementation of the assay the hydrazine indicator conjugate 124 absorbs light at wavelengths exceeding 600 nm and emits further in the red. The cyanine dye cy5.5 is an example of such a fluorophore. Other examples of red-emitting fluorescent or luminescent indicators 122 include Alexa Fluor (633 647 660 and 680), allophycocyanin (APC), APC-Cy7, Cy7, Bodipy (630/650-X, 650/665-X, 665/676), Thiadicarbocyanine, TO-PRO-3, TO-PRO-5, TOTO-3, YOYO-3, YO-PRO-3, Q-Dots 650, and others.

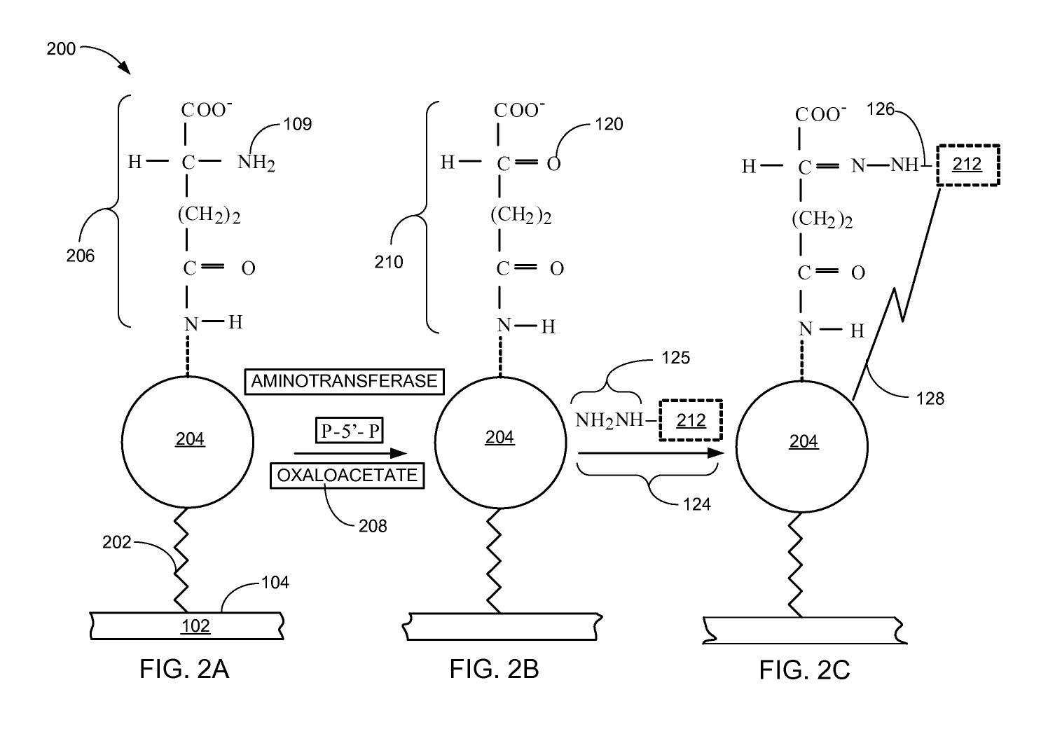

Referring now to FIGS. 2A, 2B, and 2C, therein is shown a diagram of a system for assay of aminotransferase 200, in an alternative embodiment of the present invention. The diagram of the system for assay of aminotransferase 200 depicts the platform 102 having the contact surface 104, a spacer linkage 202 immobilizes a luminescent nanocrystal 204, such as a quantum dot, on the contact surface 104. The spacer linkage 202 is optional since the luminescent nanocrystal 204 may be immobilized on the contact surface 104.

An amino acid group 206, such as L-glutamate, is coupled to the luminescent nanocrystal 204. A solution including the aminotransferase enzyme 118, acting to catalyze the transamination, an amine acceptor 208, such as oxaloacetate or pyruvate, and pyridoxal-5′-phosphate (P-5′P), a protein derived from vitamin B6, is required as a co-enzyme for both ALT and AST transamination.

Referring now to FIG. 2B, therein is shown a diagram of the system for assay of aminotransferase 200, in an intermediate step of the alternative embodiment of the present invention. The diagram of the system for assay of aminotransferase 200 depicts the luminescent nanocrystal 204 with one of the products of the aminotransferase catalyzed reaction 210, having the ketone group 120, in place of the amine side-group 109. As a second step, the luminescent nanocrystal 204, with the ketone group 210, is exposed to a hydrazine indicator conjugate 124 containing a bioluminescent molecule 212 with a hydrazine side group 125, NH2NHR, where R denotes a molecule of the bioluminescent molecule 212. The bioluminescent molecule 212 may be a mutant of Renilla reniformis luciferase, also known as Luc8.

Referring now to FIG. 2C, therein is shown a diagram of the system for assay of aminotransferase 200, in a final step of the alternative embodiment of the present invention. The diagram of the system for assay of aminotransferase 200 depicts the ketone group 210 having reacted with the hydrazine indicator conjugate 124, forming the hydrazone bond 126, and placing the indicator 122 in close proximity (within the Foster distance) to the luminescent nanocrystal 204. The indicator 122 that remains unbound produces the light emission 128 in the blue to green spectrum having a wavelength of approximately 450-550 nm, while the indicator 122 that is bound to the luminescent nanocrystal 204 transfers energy to the luminescent nanocrystal 204 to provide emission in a red to infrared spectrum having a wavelength in the range 600-900 nm. Using a simple long-wavelength of pass-band filter, or a monochromator, the light emission 128 emitted at 600 nm to 900 nm (indicative of the amount of bound Luc8) can be separated from the shorter wavelength emission. The bioluminescence resonance energy transfer (BRET) emission from the luminescent nanocrystal 204 can then be collected independently from indicator 122 that may be unbound in the background.

The inventive approach described in this patent may be implemented in any format including microscope slides, arrays, vessels, well plates, microfluidic channels, filters, porous materials and any combination thereof.

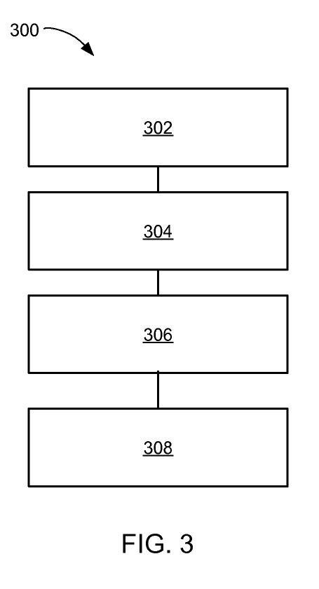

Referring now to FIG. 3, therein is shown a flow chart of a system for assays of aminotransferase 300 for operating the system for assays of aminotransferase in an embodiment of the present invention. The system 300 includes providing a platform having a contact surface in a block 302; immobilizing an amino acid group, having an amine side group, on the contact surface in a block 304; transforming the amine side group, in the amino acid group to a ketone group in a block 306; and reacting an indicator with the ketone group for displaying a light emission from the indicator in a block 308.

In greater detail, a system for assays of aminotransferase according to an embodiment of the present invention, is performed as follows:
1. Providing a platform having a contact surface including providing an attaching site on the contact surface. (FIG. 1)
2. Immobilizing an amino acid group, having an amine side group, on the contact surface including a spacer molecule between the attaching site and the amino acid group. (FIG. 1)
3. Transforming the amine side group, in the amino acid group, to a ketone group through an amine transfer reaction catalyzed by the aminotransferase enzyme. (FIG. 1) and
4. Reacting an indicator with the ketone group for displaying a light emission from the indicator including forming a hydrazone bond between the ketone group and the indicator. (FIG. 1)

It has been discovered that the present invention thus has numerous aspects.

A principle aspect is that the present invention may be implemented in any format including microscope slides, arrays, well plates, microfluidic channels, filters, porous materials and any combination thereof.

Another aspect is the present invention provides a light emission in the red spectrum making the detection and differentiation of bound indicator easier to detect due to the longer wavelength.

Yet another important aspect of the present invention is that it valuably supports and services the historical trend of reducing costs, simplifying systems, and increasing performance.

These and other valuable aspects of the present invention consequently further the state of the technology to at least the next level.

Thus, it has been discovered that the system for assays of aminotransferase of the present invention furnishes important and heretofore unknown and unavailable solutions, capabilities, and functional aspects for detecting and quantifying the amount of aminotransferase in complex biological fluids, such as blood, serum, and plasma. The resulting processes and configurations are straightforward, cost-effective, uncomplicated, highly versatile and effective, can be surprisingly and unobviously implemented by adapting known technologies, and are thus readily suited for efficiently and economically manufacturing assay devices for the detection and quantification of aminotransferase. The resulting processes and configurations are straightforward, cost-effective, uncomplicated, highly versatile, accurate, sensitive, and effective, and can be implemented by adapting known components for ready, efficient, and economical manufacturing, application, and utilization.

While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters hithertofore set forth herein or shown in the accompanying drawings are to be interpreted in an illustrative and non-limiting sense.

1. An assay method comprising:
providing a platform having a contact surface;
immobilizing an amino acid group, having an amine side group, on the contact surface;
transforming the amine side group, in the amino acid group to a ketone group; and
reacting an indicator with the ketone group for displaying a light emission from the indicator.

2. The method as claimed in claim 1 wherein transforming the amino group to the ketone group includes catalyzing by an aminotransferase enzyme.

3. The method as claimed in claim 1 wherein reacting the indicators includes mixing a hydrazine indicator conjugate with the ketone group.

4. The method as claimed in claim 1 wherein displaying the light emission includes providing the light emission in a red to infrared spectrum.

5. The method as claimed in claim 1 further comprising immobilizing a luminescent nanocrystal on the contact surface.

6. An assay system comprising:
a platform having a contact surface;
a ketone group immobilized on the contact surface; and
an indicator linked with the ketone group for displaying a light emission from the indicator.

7. The system as claimed in claim 6 wherein the ketone group immobilized on the contact surface includes a spacer molecule between the contact surface and the amino acid group.

8. The system as claimed in claim 6 wherein the indicator includes a chemiluminescent molecule or a bioluminescent molecule.

9. The system as claimed in claim 6 wherein the light emission provides a red to infrared light.

10. The system as claimed in claim 6 further comprising a luminescent nanocrystal immobilized on the contact surface.

11. The system as claimed in claim 6 further comprising:
an attaching site on the contact surface; and
a hydrazone bond between the ketone group and the indicator.

12. The system as claimed in claim 11 wherein the ketone group immobilized on the contact surface includes a carboxylate group between the contact surface and the ketone group.

13. The system as claimed in claim 11 wherein the indicator includes a chemiluminescent molecule or a bioluminescent molecule bonded to a luminescent nanocrystal.

14. The system as claimed in claim 11 wherein the light emission provides a red to infrared spectrum having an emission wavelength in the range of 600 nm to 900 nm.

15. The system as claimed in claim 11 further comprising a luminescent nanocrystal immobilized on the contact surface includes a hydrazone bond between the indicator and the luminescent nanocrystal.

16. An assay method comprising:
providing a platform having a contact surface including providing an attaching site on the contact surface;
immobilizing an amino acid group, having an amine side group, on the contact surface including bonding a carboxylate group between the attaching site and the amino acid group;
transforming the amine side group, in the amino acid group to a ketone group by catalyzing with an aminotransferase enzyme; and
reacting an indicator with the ketone group for displaying a light emission from the indicator including forming a hydrazone bond between the ketone group and the indicator.

17. The method as claimed in claim 16 wherein catalyzing with an aminotransferase enzyme includes mixing with aspartate aminotransferase or alanine aminotransferase.

18. The method as claimed in claim 16 wherein reacting the indicator includes mixing a hydrazine indicator conjugate with the ketone group including reacting a hydrazine side group linked to the indicator for forming the hydrazone bond.

19. The method as claimed in claim 16 wherein displaying the light emission includes providing the light emission in a red to infrared spectrum including sourcing the light emission having a wavelength in the range of 600 nm to 900 nm.

20. The method as claimed in claim 16 further comprising immobilizing a luminescent nanocrystal on the contact surface including immobilizing a quantum dot having the hydrazone bond to the indicator for generating the light emission in a red to infrared spectrum by bioluminescence resonance energy transfer.
